(19) United States
(12) Patent Application Publication
(10) Pub. No.: US 2015/0088012 A1
(43) Pub. Date: Mar. 26, 2015

(54) Light Assembly

(71) Applicant: 3 Sticks Pty Ltd, Mosman (AU)

(72) Inventor: Chauncey Hammond, Mosman (AU)

(21) Appl. No.: 14/381,837

(22) PCT Filed: Mar. 11, 2013
(86) PCT No.: PCT/AU2013/000237

§ 371 Date: Aug. 28, 2014

(30) Foreign Application Priority Data
Mar. 12, 2012 (AU) 2012900979
Oct. 31, 2012 (AU) 2012904790

(51) Int. Cl. F21V 33/00; F21V 21/14; A61B 5/024; F21L 4/08; H05K 5/00; G01S 19/42; F21L 4/00; F21K 99/00

(52) U.S. Cl. CPC F21V 33/0004; F21L 4/00; F21V 21/14; F21K 9/30; F21L 4/08; H05K 5/0086; G01S 19/42; A61B 5/02438; F21Y 2101/02
USPC 600/500; 362/189; 362/183; 361/679.01; 342/357.25

(57) ABSTRACT

A light assembly to be worn by a user. Typically the light assembly (10) would be worn on a user's arm. The assembly (10) includes a body (11) of a “U” shaped configuration so as to have a base (14) extending between a pair of generally co-extensive extensive arms (12). The body (11) is formed of resilient material so as to be retained in position when urged into engagement with an object, which is preferably a users arm or leg, by resilient deformation of the body. At least one light, which is preferably an LED module (15), is mounted in the body (11).

### FIELD

The present invention relates to light assemblies and more particularly but not exclusively to light assemblies to be attached to the arm or a leg of a user.

### BACKGROUND

The visibility of pedestrians and riders is important, particularly after sunset. Although it is known to provide joggers, pedestrians and riders with lights, a disadvantage of these known lights is that they are not easily secured relative to the user. A further disadvantage is that these lights are only effective in a forward (or reverse) direction.

It is also known to provide joggers and others exercising with heart rate monitors. It is also known to have in mobile form, such as mobile telephones, GPS functions.

A disadvantage of the above discussed devices is that they provide a single function thereby requiring the user to carry several of the devices where more than one function is required.

### OBJECT

It is the object of the present invention to overcome or substantially ameliorate the above disadvantage.

### SUMMARY

There is disclosed herein a light assembly including:

a body of “U” shaped configuration so as to encompass an area within which an object is to be located, the body being formed of resilient material so as to be retained in position secured to the object by being urged into engagement therewith by resilient deformation of the body;

at least one light mounted in the body; and

a battery mounted in the body and electrically coupled to the light to cause illumination of the light.

Preferably, the body is moulded of plastics material. In an alternative form the body is moulded of natural or synthetic rubber.

Preferably, the body has a pair of arms joined by a base, and there are at least two lights, each light being mounted in a respective one of the arms.

Preferably, each light is a light assembly so as to include an LED module with a battery.

In an alternative preferred form, each light is an LED module, and a battery is mounted in the body and is in communication with each light.

Preferably, a switch is operable to connect the battery to each light.

Preferably, the assembly includes a USB charging port connected to the battery.

Preferably, the body is shaped to engage the leg or arm of a user so that the light assembly is secured thereto.

There is further disclosed herein an assembly having a switch, the assembly including:

a body configured so as to encompass an area within which an object is to be located so that the assembly is mounted on the object, the body including a socket;

a battery mounted in the body;

conductors operably associated with the battery and switch with the conductors extending to said socket so as to provide contacts to provide electric power; and

a module releasably secured in the socket, the module including electrical components engaged with the contacts so that the module is powered by the battery upon operation of the switch by a user.

Preferably, the switch is mounted in the body. In an alternative form each module includes a battery.

Preferably, said module is a light.

Preferably, said module includes an electronic circuit and a screen coupled to the circuit, with the circuit to provide information to be illustrated on the screen.

Preferably, the circuit provides an indication of a user's body function.

Preferably, the body function is a user's pulse.

In an alternative preferred embodiment, the electronic circuit provides a GPS function with a user's location illustrated on the screen and/or provides a signal enabling the user's location to be determined.

Preferably, the socket is a first socket, and said body provides a second socket, with the second socket also having electrical contacts to receive electric power from the battery by operation of the switch, and a further one of the modules located in the socket and an electric contact with the contacts.

### BRIEF DESCRIPTION OF THE DRAWINGS

Preferred forms of the present invention will now be described by way of example with reference to the accompanying drawings wherein:

FIG. 1 is a schematic isometric view of a light assembly;

FIG. 2 is a schematic end elevation of the light assembly of FIG. 1;

FIG. 3 is a schematic side elevation of the light assembly of FIG. 1;

FIG. 4 is a schematic isometric view of a further light assembly;

FIG. 5 is a schematic end elevation of the light of assembly of FIG. 4;

FIG. 6 is a schematic side elevation of the light assembly of FIG. 4;

FIG. 7 is a schematic isometric view of an assembly to be worn by a user;

FIG. 8 is a further schematic isometric view of the assembly of FIG. 7;

FIG. 9 is a parts exploded isometric view of the assembly of FIG. 7;

FIG. 10 is a further schematic parts exploded isometric view of the assembly of FIG. 7;

FIG. 11 is a schematic sectioned side elevation of a module employed in the assembly of FIG. 7;

FIG. 12 is a schematic isometric view of a modification of the assemblies 10 and 20; and

FIG. 13 is a schematic sectioned side elevation of the assembly of FIG. 12.

### DETAILED DESCRIPTION OF THE PREFERRED EMBODIMENTS

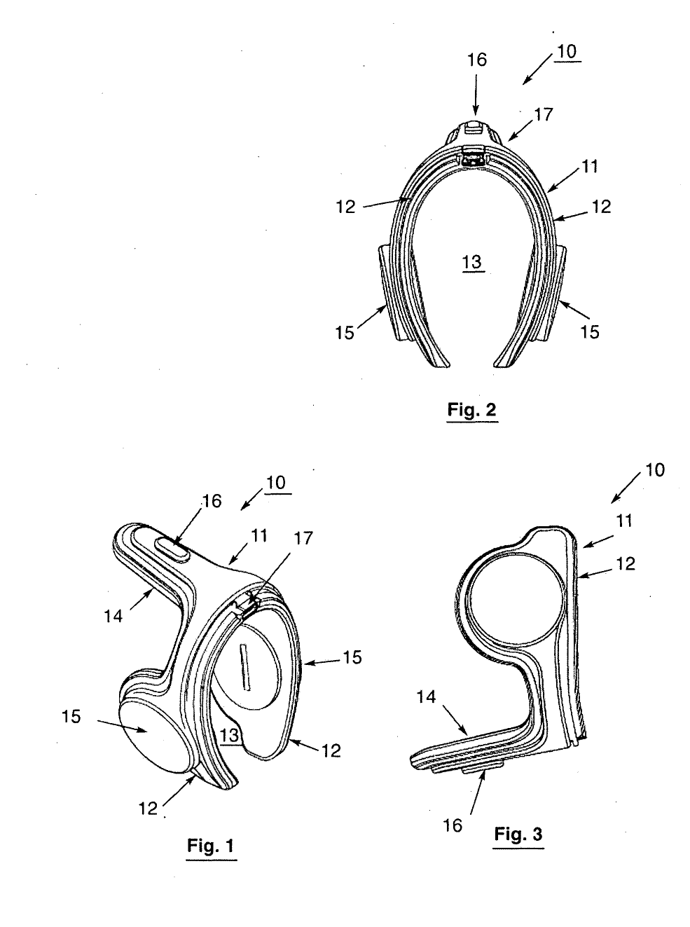

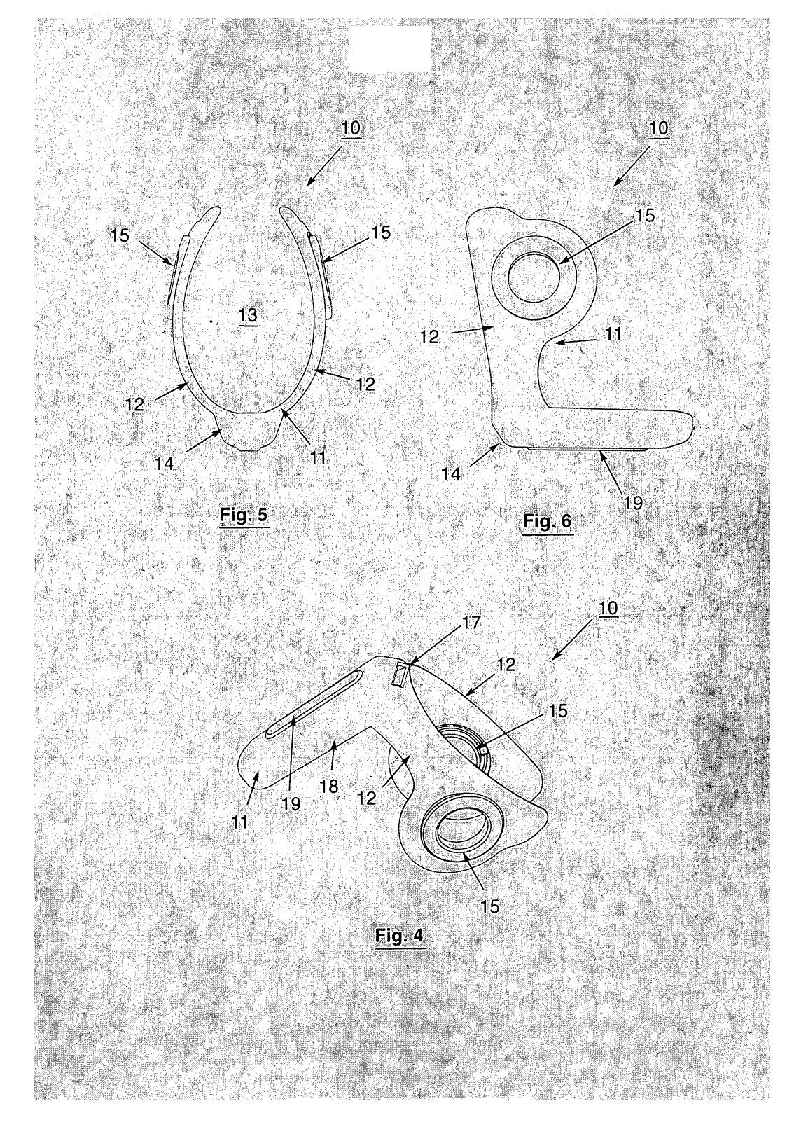

In FIGS. 1, 2 and 3 of the accompanying drawings there is schematically depicted a light assembly 10. The assembly 10 includes a body 11 formed of plastics material. The body 11 is of a “U” configuration so as to have a pair of generally coextensive arms 12 that are generally arcuate so as to be concave outwardly away from each other, and so as to encompass an area 13. The arms 12 extend from a base 14 that extends generally normal to the arms 12.

Each of the arms 12 is provided with an LED module 15 that includes a battery and LEDs. The battery would be connected to the LEDs by means of a manual switch 16. The LEDs may be of different colours.

The body 11 is also provided with a USB charging port 17 via which the batteries may be charged. As an alternative, the port 17 may be adapted to engage a charger.

The above body 11 is formed of plastics material so as to be resiliently deformable. The arms 12 are shaped to pass about the leg or arm of a user so as to be resiliently deformed and therefore urged back into contact with the user so as to secure the assembly 10 in position. Preferably, the body 11 is coated with a “soft” material such as silicon so that it is comfortable to wear.

As a particular example, the assembly 10 could be fixed to a user's arm for the purposes of making the user more visible, while the user may also use the assembly 10 to indicate an intended direction of travel. In particular the assembly 10 would improve the user's side visibility.

The LED modules 15 face outwardly in opposite directions from the arms 12.

The body 11 may also include sensors that detect a user's pulse, movement, temperature, etc with the information recorded for later use. The body 11 may also incorporate a GPS function.

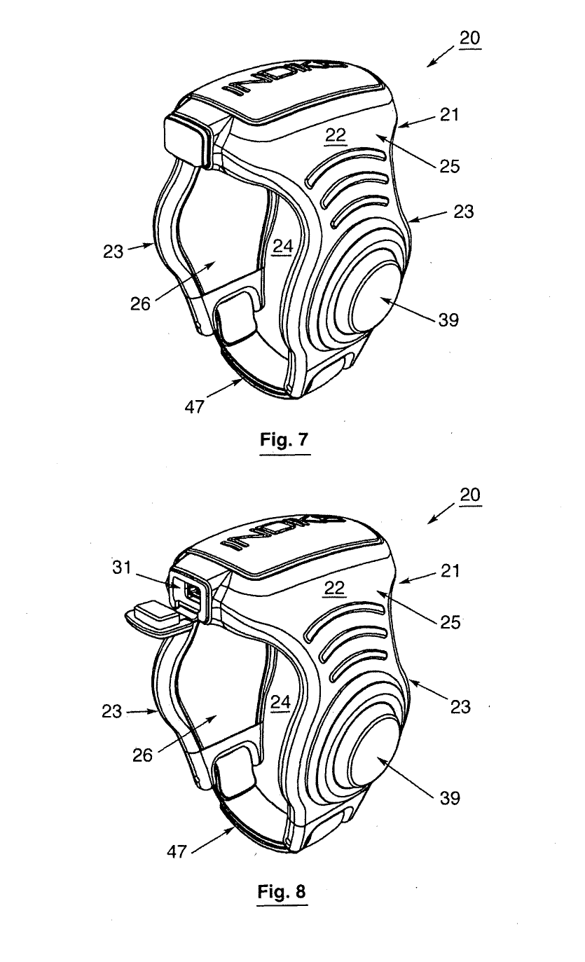

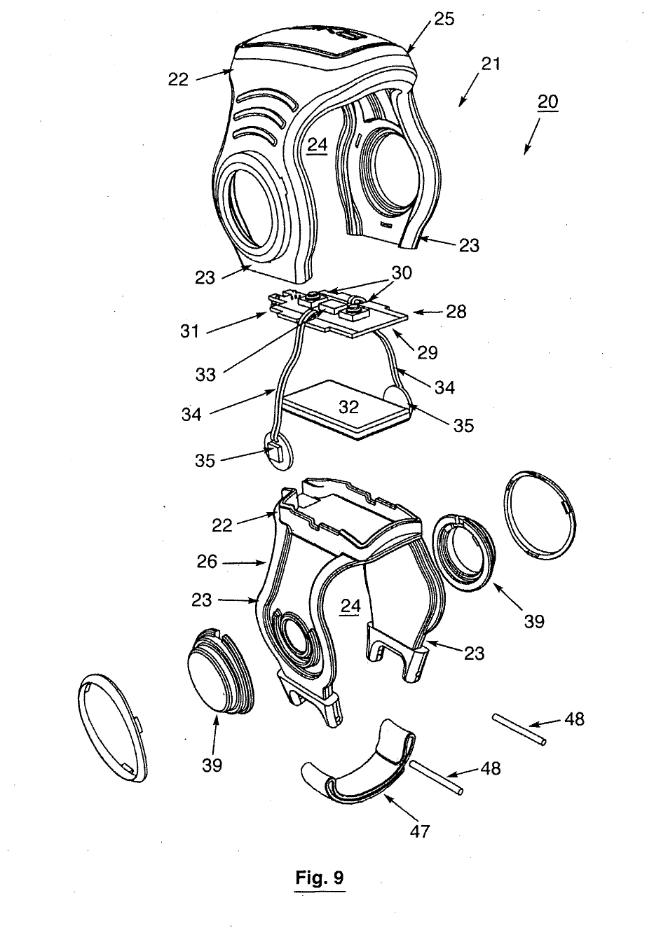

In the embodiment of FIGS. 4, 5 and 6, the base 14 is provided with a higher capacity LiPo battery 18 and a switch 19 that connects the battery 18 to the LED modules 15. In this embodiment, the modules 15 would not include a battery.

The module 15 may include a “chip” so that the module 15 is programmable to produce a desired light sequence. In an alternative embodiment, the “chip” may be included in the body 11.

The module 15 is preferably replaceable or interchangeable.

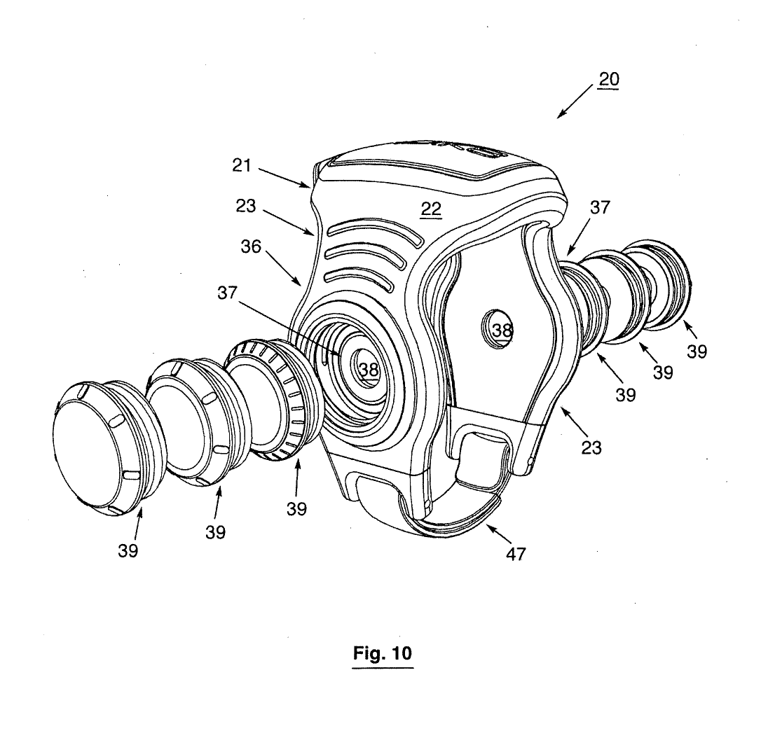

FIGS. 7 to 11 illustrate an assembly 20 to be worn by a user. Typically the assembly 20 would be mounted on a user's arm.

The assembly 20 includes a body 21 of a “U” shaped configuration so as to have a base 22 extending between a pair of generally parallel co-extensive arms 23. Preferably the body 21 is moulded of plastics material so that it can be resiliently deformed. The body 21 encompasses an area 24 within which an object (typically the wearer's arm) is located so that the arms 23 are moved apart so as to be resiliently urged inward so as to securely mount the assembly 20 on the user's arm. That is the user's arm is clamped between the arms 23.

The body 21 includes an outer portion 25 fixed to an inner portion 26, with the portions 25 and 26 providing a cavity 27 within which electronic components 28 are housed and secured in position. The components 28 include a board 29 upon which there is mounted a pair of switches 30, with the board 29 also providing an electric circuit providing a port 31, typically a USB port.

Also mounted in the cavity 27 is a battery 32, with the battery 32 coupled to the port 31 to provide full charging of the battery 32. The battery 32 would also be coupled to the switches 30, the port 31, battery 32 and an integrated circuit 33.

Also connected to the electric components 28 (integrated circuit 33) are wires 34 that extend to conductors 35 so that upon operation of the switches 36, electric power is delivered to the conducts 35.

Preferably, the outer portion 25 and inner portion 26 snap engage so as to be secured together but are releasable to expose the battery 32 for replacement purposes.

The body 21 provides a pair of sockets 36 which in this embodiment are of a cylindrical configuration so as to be surrounded by a threaded section 37. The inner end of the socket 37 has an aperture 38 within which the contacts 35 are located.

Releasably mounted in each socket is a module 39. In FIG. 10 several of the modules 39 are illustrated. However it should be appreciated that on one side of the assembly 10 one module 39 may be employed while on the other side of the assembly 10 a different module 39 may be employed. The module 39 is configured to provide a specific function or functions. These functions can include storing and transmitting information relating to the wearer.

The modules 39 provide a variety of functions. For example, one module 39 may merely provide a light. As an alternative one of the modules 39 may provide a GPS function. While further another module 39 might provide a read out of data relative to the wearer, such as a pulse, body temperature and blood oxygen level, as examples. As a further alternative, the module 39 may provide a stop watch function.

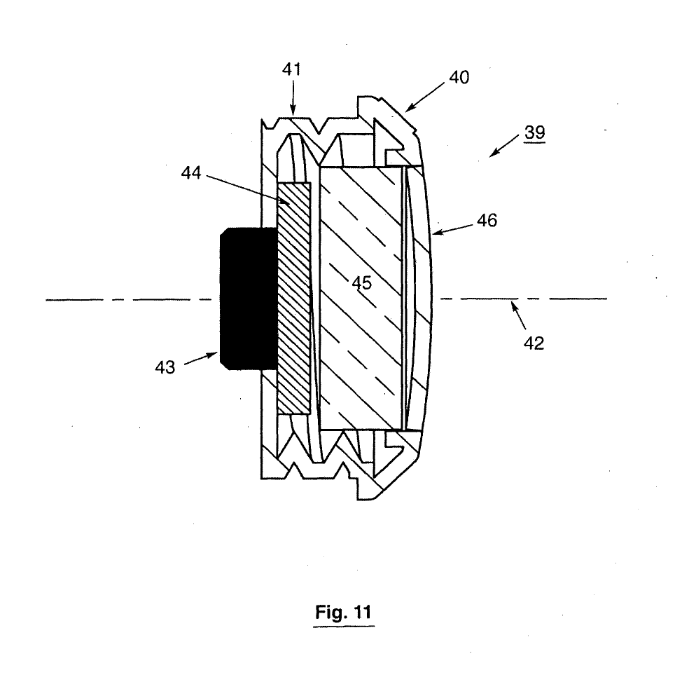

In FIG. 11 there is schematically depicted an example of a module 39. In this particular example, the module 39 includes a hollow body 40 having a threaded section 41 that threadably engages the section 37 so that upon rotation of each module 39 about the central axis 42 within one of the socket 36, relative to the body 21, the module 39 is either secured in or removed from within the associated socket 36.

The module 39 of FIG. 11 includes a contact portion 43 having contact areas that engage the contacts 35 so that the module 39 is electrically connectable to the battery 32 via the switches 30 and integrated circuit 33. The module 39 also includes an electric circuit 44 that might provide a GPS function or electronic circuitry that can detect a user's pulse, or other function as described above. A screen 45 is electrically coupled to the circuit 44 so as to receive signals therefrom, with the screen 45 then providing a display of information such as a user's pulse. A transparent protective layer 46 secured to the body 44 and overlays the screen 45.

The GSP function may include a display illustrating a location and/or transmit a signal for the purposes of providing information in respect of the user's location.

As an alternative embodiment, the circuit 44 and screen 45 may merely be replaced with a light assembly.

Preferably, the assembly 20 further includes a strap 47 secured to the body 21 by pins 48. Preferably, the strap 47 would be adjustable in length to aid in securing the assembly 20 to an object, such as a user's arm.

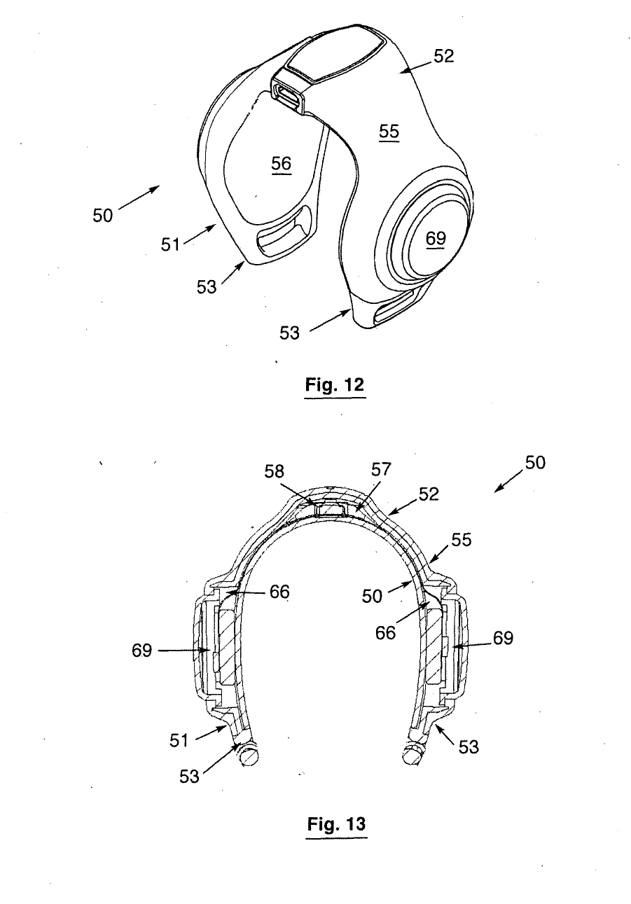

In the embodiment of FIGS. 12 and 13, like the embodiments of FIGS. 7 to 11, the assembly 50 includes a body 51 of a “U” shaped configuration so as to have a base 52 extending between a pair of generally parallel co-extensive arms 53. Preferably the body 51 is moulded of plastics material so that it can be resiliently deformed. The body 51 encompasses an area 54 within which an object (typically the user's arm) is located so that the arms 53 are moved apart so as to be resiliently urged inwardly so as to be securely mounted on the user's arm.

The body 51 includes an outer portion 55 an end portion 56, with the portions 55 and 56 providing a cavity 57 within which electronic components 58 are housed and secured in position. The components 58 may be the same as the components 28 of the previous embodiment.

Like the previous embodiment, the body provides a pair of sockets 36. Like the previous embodiment, these sockets 66 are of a cylindrical configuration so as to receive modules 69. The modules 69 may be as previously described and may be identical to the modules 39.

In the above embodiments, the battery 32 is located in the body 21. However a battery 32 could be provided, as an alternative, in each module 39.

Preferably, the assemblies 10, 20 and 50 are waterproof, including the modules 15, 39 and 69.

In the above embodiments, the switches, such as the switch 30, may be a mechanical or electrical switch or maybe motion activated. In a further alternative preferred form, the switch may be remote from the assembly.

As an alternative to a USB port to charge the battery or batteries, each battery may be inductively charged. Other charging means such as a pin or “jack” or coaxial connectors may be used. As a further alternative, movement of the assemblies 10, 20 and 50 may be used to generate a charge to operate the assemblies 10, 20 and 50, including charging the batteries.

1-16. (canceled)

17. A light assembly including:
a body of “U” shaped configuration so as to encompass an area within which an object is to be located, the body being formed of resilient material so as to be retained in position secured to the object by being urged into engagement therewith by resilient deformation of the body;
at least one light mounted in the body; and
a battery mounted in the body and electrically coupled to the light to cause illumination of the light.

18. The light assembly of claim 17, wherein the body is moulded of plastics material.

19. The light assembly of claim 17, wherein the body has a pair of arms joined by a base, and there are at least two lights, each light being mounted in a respective one of the arms.

20. The light assembly of claim 18, wherein each light is a light assembly so as to include an LED module with a battery.

21. The light assembly of claim 17, wherein each light is an LED module, and a battery is mounted in the body and is in communication with each light.

22. The light assembly of claim 17, wherein a switch is operable to connect the battery to each light.

23. The light assembly of claim 17, wherein the assembly includes a USB charging port connected to the battery.

24. The light assembly of claim 17, wherein the body is shaped to engage the leg or arm of a user so that the light assembly is secured thereto.

25. The light assembly of claim 17, wherein the body has a pair of arms joined by a base, and there are at least two lights, each light being mounted in a respective one of the arms, with each light being a light assembly including an LED module with a battery.

26. The light assembly of claim 18, wherein each light is an LED module, and a battery is mounted in the body and is in communication with each light, with a switch operable to connect the battery to each light.

27. The light assembly of claim 18, wherein the body is moulded from synthetic or natural rubber.

28. An assembly having a switch, the assembly including:
a body configured so as to encompass an area within which an object is to be located so that the assembly is mounted on the object, the body including a socket;
a battery mounted in the body;
conductors operably associated with the battery and switch with the conductors extending to said socket so as to provide contacts to provide electric power; and
a module releasably secured in the socket, the module including electrical components engaged with the contacts so that the module is powered by the battery upon operation of the switch by a user.

29. The light assembly of claim 28, wherein the switch is mounted in the body. In an alternative form each module includes a battery.

30. The light assembly of claim 28, wherein said module is a light.

31. The light assembly of claim 28, wherein said module includes an electronic circuit and a screen coupled to the circuit, with the circuit to provide information to be illustrated on the screen.

32. The light assembly of claim 31, wherein the circuit provides an indication of a user's body function.

33. The light assembly of claim 32, wherein the body function is a user's pulse.

34. The light assembly of claim 31, wherein the electronic circuit provides a GPS function with a user's location illustrated on the screen and/or provides a signal enabling the user's location to be determined.

35. The light assembly of claim 28, wherein the socket is a first socket, and said body provides a second socket, with the second socket also having electrical contacts to receive electric power from the battery by operation of the switch, and a further one of the modules located in the socket and an electric contact with the contacts.

36. The light assembly of claim 34, wherein the socket is a first socket, and said body provides a second socket, with the second socket also having electrical contacts to receive electric power from the battery by operation of the switch, and a further one of the modules located in the socket and an electric contact with the contacts.
